(19) United States
(12) Patent Application Publication
(10) Pub. No.: US 2006/0025841 A1
(43) Pub. Date: Feb. 2, 2006

(54) Thalamic stimulation device

(76) Inventor: Cameron McIntyre, Cleveland, OH (US)

Correspondence Address: KENYON & KENYON, 1500 K STREET NW, SUITE 700, WASHINGTON, DC, 20005, US

(21) Appl. No.: 11/191,867

(22) Filed: Jul. 27, 2005

Related U.S. Application Data
(60) Provisional application No. 60/591,210, filed on Jul. 27, 2004.

(51) Int. Cl. A61N 1/05

(52) U.S. Cl. 607/116

(57) ABSTRACT

The present invention provides an electrical lead configured for stimulation of the thalamus. The electrical lead provides for preferential stimulation of the medial or lateral thalamus as well as specific nuclei within the thalamus, such as the intralaminar nuclei.

### CROSS-REFERENCE TO RELATED APPLICATIONS

The present application claims priority to U.S. Provisional Application No. 60/591,210, filed on Jul. 27, 2004, which is incorporated by reference herein.

### FIELD OF THE INVENTION

The present application is directed to an implantable electrical stimulation device configured to stimulate target sites of the thalamus.

### BACKGROUND OF THE INVENTION

Deep brain stimulation is becoming an increasingly preferred form of therapy for certain neurological conditions and disorders. Such therapy provides distinct advantages over surgical lesioning techniques, which are still being used to affects disorders such as Parkinson's disease, essential tremor, and dystonia. In particular, unlike surgical lesioning techniques, electrical stimulation is a reversible and adjustable procedure that provides continuous benefits as the patient's disease progresses and the patient's symptoms evolves. Currently, deep brain stimulation of the subthalamic nucleus and the globus pallidus interna is approved for treatment of Parkinson's disease and deep brain stimulation of the ventral intermediate nucleus is approved for treatment of essential tremor. Other target sites in the brain to treat additional disorders are also contemplated. For example, as described in U.S. Pat. No. 5,938,688 and U.S. Pat. No. 6,167,311, respectively, the intralaminar nuclei of the thalamus could be stimulated to treat patients with impaired cognitive function and/or patients with psychological disorders.

Current electrical leads used in deep brain stimulation, however, do not provide precise targeting of the areas of the thalamus such as the intralaminar nuclei, such that the desired volume of tissue is stimulated. Accordingly, there is a need in the art for a stimulation device that precisely targets specific regions of the thalamus, maximizing stimulation of these specific regions and minimizing stimulation of adjacent tissue that results undesirable side effects.

### SUMMARY OF THE INVENTION

In an embodiment, the present invention provides a lead comprising a body having electrodes thereon. Preferably, the lead has any one of, all of, or any combination of the following features: the body having a diameter of between about 0.75 to about 1.25 millimeters (mm); the body having between about 4 to about 6 electrodes; electrodes that span about 110 to about 170 degrees about the body; and at least one of and preferably all of the electrodes having a height of between about 3 to about 4 millimeters.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a perspective view of a lead according to an embodiment of the present invention.

FIG. 2 is a schematic illustration of the volume of activation generated by an electrical lead of the present invention placed in the brain.

FIG. 3 is a schematic illustration of the volume of activation generated by an electrical lead of the present invention placed in the brain.

FIG. 4 is a schematic illustration of the volume of activation generated by an electrical lead of the present invention placed in the brain.

FIG. 5 is a schematic illustration of the volume of activation generated by an electrical lead of the prior art placed in the brain.

FIG. 6 is a diagrammatic view of a patient in which an embodiment of a lead according to the present invention has been implanted.

### DETAILED DESCRIPTION OF THE INVENTION

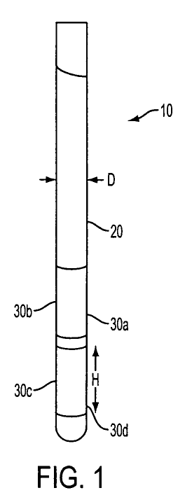

Referring to FIG. 1, in an embodiment, the present invention provides an electrical lead 10 comprising a body 20 having electrodes 30 thereon. Preferably, electrical lead 10 has any one of, all of, or any combination of the following features: body 20 has a diameter D of between about 0.75 to about 1.25 millimeters (mm); body 20 has about 4 to about 6 electrodes 30 thereon; electrodes 30 each span about 110 to about 170 degrees about body 20; and at least one of and preferably all of electrodes 30 have a height H of between about 3 to about 4 mm.

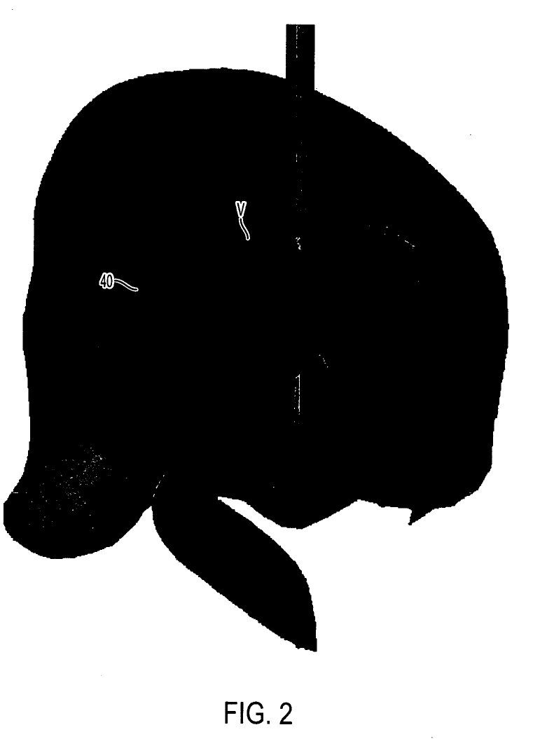

Electrical lead 10 can be used to stimulated specific regions of the thalamus to provide precise directional stimulation of such specific regions. For example, referring to FIG. 2, which is a schematic illustration of electrical stimulation of regions of the thalamus by electrical lead 10 positioned in the thalamus, activation of electrode 30b results in a volume of activation V that reaches the intralaminar nuclei as well as parts of the lateral thalamus 40. Such volume of activation is generated by -−3V, 90 microsecond, and approximately 50 hertz stimulation.

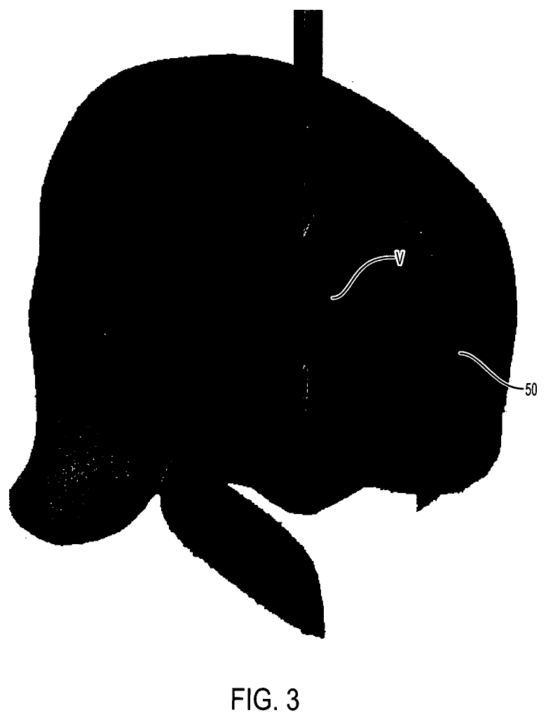

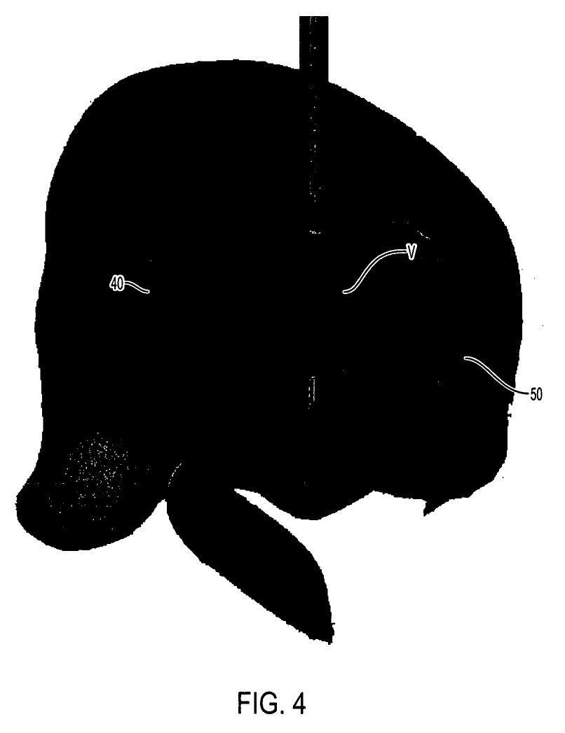

Referring to FIG. 3, under the same stimulation parameters, activation of electrode 30a results in a volume of activation V that reaches the intralaminar nuclei as well as parts of the central medial nucleus of the medial thalamus 50. Referring to FIG. 4, activation of electrodes 30a and 30b results in a volume of activation V that reaches the intralaminar nuclei as well as parts of the medial thalamus 50 and lateral thalamus 40. Accordingly, depending on the particular therapeutic application, different electrodes 30 and different combinations of electrodes 30 of electrical lead 10 can be activated to provide different directional stimulation of specific regions of the thalamus, such as the lateral thalamus or the medial thalamus as well as nuclei within the lateral or medial thalamus, such as the intralaminar nuclei. Of course, electrical lead 10 is also capable of stimulating both the lateral and medial thalamus.

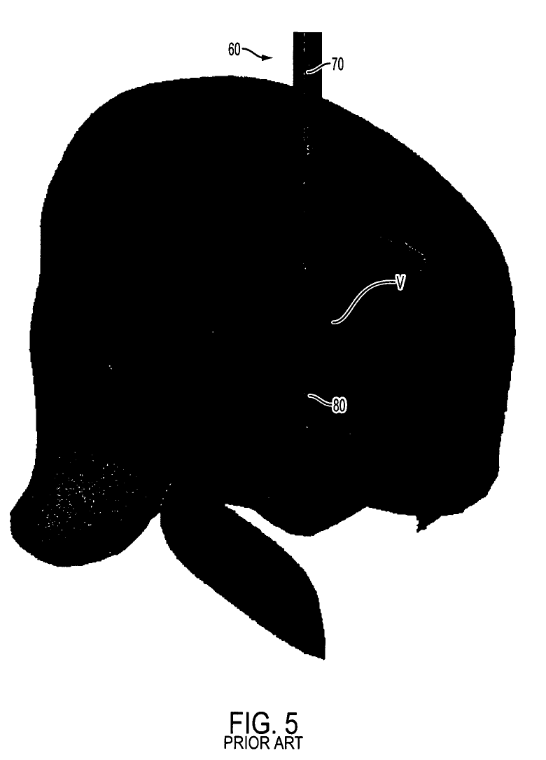

FIG. 5 is a schematic illustration of electrical stimulation of regions of the thalamus by a prior art electrical lead 60 positioned in the thalamus that has shorter and thicker electrodes 80 (four electrodes 80 in all) that each span 360 degrees about body 70 under the same stimulation parameters as used for stimulation depicted in FIGS. 1-4 (−3V, 90 microsecond, and approximately 50 hertz stimulation). Specifically, the upper two electrodes 80 of lead 60 are activated. As seen from a comparison of FIG. 4 and FIG. 5, electrical lead 10 of the present invention can generate the same shape of activation V as can prior art lead 60, but for given stimulation parameters, stimulation by electrical lead 10 of the present invention results in a greater volume of activation V than the volume of activation V generated by prior art leads. Electrical leads 10 of the present invention also provide additional flexibility in allowing for preferential stimulation of the lateral or medial thalamus.

Although not limited to any particular areas of the thalamus, the electrical lead of the present invention is particularly useful for stimulating the intralaminar nuclei, which include, for example, the centromedial nucleus, the parafascicular nucleus, the paracentral nucleus, the central lateral nucleus, and the central medial nucleus. The electrical lead 10 may also be used for preferential stimulation of one side or the other side of nuclei or a nucleus split by the internal medullary lamina.

Electrodes 30 of the present invention can be adjustably powerable. For example, the pulsing parameters of the electrodes 30 may be adjusted to initiate, stop, increase, or decrease the pole combinations, energy, amplitude, pulse width, waveform shape, frequency, and/or voltage or any other pulsing parameter known to one of skill in the art to adjust the degree of stimulation delivered thereby. In a preferred embodiment, each electrode 30 of body 20 of lead 10 is selectively powerable such that the pulsing parameters of an electrode 30 can be adjusted independent of the pulsing parameters of another electrode 30.

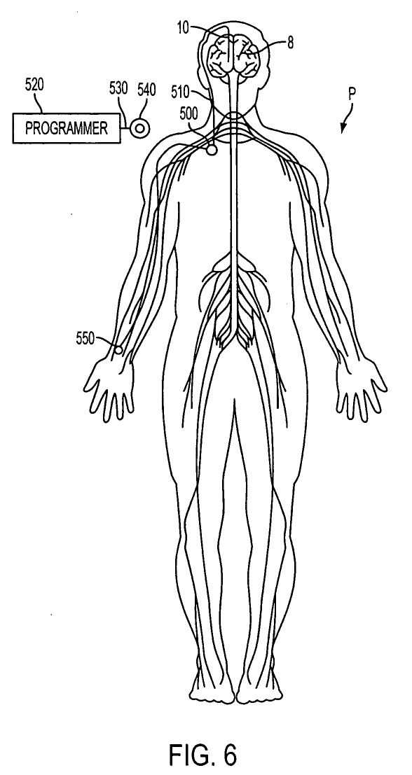

Referring to FIG. 6, the selective powerability over each electrode 30 may be achieved by employed a system including a programmer 520 coupled via a conductor 530 to a telemetry antenna 540. The programmer 520 is capable of sending signals via the telemetry antenna 540 to control the electrical signal delivered to electrodes 30 and, optionally, to control mechanism 100, in embodiments where control mechanism 100 is remotely operated. Such a system permits the selection of various pulse output options after lead 10 is implanted using telemetry communications. The present invention also contemplated radio-frequency systems to selectively power electrodes 30.

As will be understood by one of skill in the art, the independent powerability of electrodes 30 also provides a practitioner with another means of modify or steering the direction of stimulation as the locus of stimulation can be selectively adjusted to precisely target portions of the thalamus to achieve the desired therapy. For example, electrode 30a may be powered to stimulate an area adjacent thereto while the signal to electrode 30c may be substantially minimized to reduce or stop stimulation to an area adjacent to electrode 30c. Because the locus of stimulation can be selectively adjusted and/or steered in this embodiment of lead 10, specific areas of the thalamus can be precisely targeted to achieve the desired therapy. Other or additional means of selectively steering electrical stimulation may also be utilized in the present invention, such as the methods described in U.S. Pat. No. 5,713,922, which is incorporated by reference herein.

A neural stimulation delivery system including lead 10 to stimulate neural tissue to affect a neurological condition may include other components useful in identifying, monitoring, or affecting a specific thalamic site or a particular neurological condition associated with the specific thalamic site. For example, such a system could include a component for lesioning and temperature monitoring, and/or a component that has a fiberoptic monitor which allows telemetric intracranial monitoring capabilities, and/or a microelectrode recording component, and/or a sensing component to incorporate a feedback mechanism to assist in determining whether lead 10 should be adjusted. With respect to a sensing component, referring to FIG. 6, a sensor 550 can be incorporated with a system of stimulating the thalamus according to the present invention. Sensor 550 can be used with a closed-loop feedback system in order to automatically determine the level of stimulation necessary to provide the desired therapy. Sensor 550 may be implanted into a portion of a patient P's body suitable for detecting characteristics, symptoms or attributes of the condition or disorder being treated such as electrical brain activity, cerebral blood flow, and/or vital signs or other chemical and electrical activity of the body. Sensors suitable for use in a system according to the present invention include, for example, those disclosed in U.S. Pat. No. 5,711,316, which is incorporated by reference herein. In cases where the attribute of the symptom is the electrical activity of the brain, stimulating electrodes may be intermittently used to record electrical activity. Alternatively, one or more electrodes implanted within the brain may serve as a sensor or a recording electrode. When necessary, these sensing or recording electrodes may deliver stimulation therapy to the thalamus. The output of an external feedback sensor may communicate with an implanted pulse generator through a telemetry down-link.

In order to advance lead 10 through a cannula, an actuator system that creates linear motion may be provided. Lead 10 may be provided within the cannula as part of the device or lead 10 may be installed during the surgical technique.

Preferably, lead 10 is capable of being bent, capable of being pre-bent such that lead 10 has a memory bend, or capable of being pre-formed into a desired shape that has memory. For example, lead 10 may be fabricated of a shape memory alloy such as nitinol.

The present invention contemplates that electrical lead 10 is not only capable of being adjusted intra-operatively, but also is capable of being adjusted post-operatively. Specifically, lead 10 positioning may be physically adjusted (advanced, retracted, or moved to a different location) in the brain post-operatively through the use of telemetry, RF signals, or other systems known in the art. The cannula need only be inserted once while lead 10 may be re-positioned in the brain tissue multiple times to reach the desired area of the brain. Further, electrodes 30 on lead 10 may be adjusted post-operatively by turning them on or off, adjusting the voltage, adjusting the frequency, and adjusting other electrical signal parameters through the use of telemetry, RF signals, or other systems known in the art. Those skilled in the art will appreciate that electrical properties of the electrodes 30 and the resulting electrical field may be varied by selectively powering individual or groups of electrodes 30 formed from or controlled by micro-electrical mechanical systems (MEMS). Moreover, MEMS actuators may drive electrodes, drug delivery catheters, sensing probes, and the like from the cannula to desired locations in an area of interest.

Furthermore, lead 10 may also be used in conjunction with brain stimulation modeling systems as described in U.S. patent application Ser. No. 10/885,982, filed on Jul. 7, 2004, entitled “Brain Stimulation Models, Systems, Devices, and Methods” (Attorney Docket. No. 01969.001US1), which is incorporated by reference herein.

The present invention may also be implemented within a drug delivery system to provide chemical stimulation utilizing a drug, pharmaceutical, or therapeutic agent. In this embodiment, the signal generator is replaced with or includes a pump and selected electrode is replaced with a catheter. The pump may be implanted below the skin of a patient and has a port into which a hypodermic needle can be inserted through the skin to inject a quantity of a liquid, such as a drug, pharmaceutical, or therapeutic agent. the liquid agent is delivered from a pump through a catheter port into a catheter. The catheter is positioned to deliver the liquid agent to specific infusion sites in the brain. Alternatively, the electrical lead may be combined with a drug delivery system to provide both chemical and electrical modulation to the thalamus.

Although the invention has been described with reference to the preferred embodiments, it will be apparent to one skilled in the art that variations and modifications are contemplated within the spirit and scope of the invention. The drawings and description of the preferred embodiments are made by way of example rather than to limit the scope of the invention, and it is intended to cover within the spirit and scope of the invention all such changes and modifications. The present invention incorporates by reference herein in its entirety U.S. patent application Ser. No. 10,602,319, filed on Jun. 24, 2003 entitled “Directional Electrode Design and Method of Using Same.”

I claim:

1. An electrical lead adapted for stimulation of the thalamus comprising:
a body having a diameter of between about 0.75 to about 1.25 millimeters and having between about 4 to about 6 electrodes, the electrodes spanning about 110° to about 170° about the body and each of the electrodes having a height of between about 3 to about 4 millimeters.

2. The electrical lead of claim 1, wherein one or more of the electrodes are selectively powerable.

4. The electrical lead of claim 1, wherein the body is pre-bent or pre-formed into a desired shape.

5. The electrical lead of claim 4, wherein the body is fabricated of a shape memory alloy.

6. A method of selectively stimulating a region of the thalamus comprising:
providing the electrical lead of claim 1;
positioning the electrical lead of claim 1 adjacent to a region of the thalamus; and
activating the electrical lead of claim 1 to stimulate the region of the thalamus.

7. The method of claim 6, wherein the region of the thalamus is the intralaminar nuclei.

8. The method of claim 6, wherein the region of the thalamus is the lateral or medial thalamus.
